(19) United States
(12) Patent Application Publication
(10) Pub. No.: US 2007/0166766 A1
(43) Pub. Date: Jul. 19, 2007

(54) METHOD AND APPARATUS FOR MASS SPECTROMETRIC IMMUNOASSAY ANALYSIS OF SPECIFIC BIOLOGICAL FLUID PROTEINS

(75) Inventors: Urban A. Kiernan, Gilbert, AZ (US); Eric E. Niederkofler, Glendale, AZ (US); Kemmons A. Tubbs, Mesa, AZ (US); Dobrin Nedelkov, Tempe, AZ (US); Randall W. Nelson, Phoenix, AZ (US)

Correspondence Address: SNELL & WILMER L.L.P. (Main), 400 EAST VAN BUREN, ONE ARIZONA CENTER, PHOENIX, AZ, 85004-2202, US

(73) Assignee: INTRINSIC BIOPROBES, INC., Tempe, AZ (US)

(21) Appl. No.: 11/692,776

(22) Filed: Mar. 28, 2007

Related U.S. Application Data
(62) Division of application No. 10/905,029, filed on Dec. 10, 2004.
(60) Provisional application No. 60/481,766, filed on Dec. 10, 2003.

(51) Int. Cl. G01N 33/53

(52) U.S. Cl. 435/7.1

(57) ABSTRACT

Presented herein are methods, devices and kits for the mass spectrometric immunoassay (MSIA) of proteins and their variants that are present in complex biological fluids or extracts. Protein and variant levels can be determined using quantitative methods in which the protein/variant signals are normalized to signals of internal reference standard species (either doped into the samples prior to the MSIA analysis, or other endogenous protein co-extracted with the target proteins) and the values compared to working curves constructed from samples containing known concentrations of the protein or variants.

### CROSS-REFERENCE TO RELATED APPLICATIONS

This application is a divisional patent application based on utility patent application entitled “METHOD AND APPARATUS FOR MASS SPECTROMETRIC IMMUNOASSAY ANALYSIS OF SPECIFIC BIOLOGICAL FLUID PROTEINS ” and having Ser. No. 10/905,029, filed Dec. 10, 2004, which claims priority to provisional patent application entitled “METHOD AND APPARATUS FOR MASS SPECTROMETRIC IMMUNOASSAY ANALYSIS OF SPECIFIC BIOLOGICAL FLUID PROTEINS ” and having Ser. No. 60/481,766, filed Dec. 10, 2003, both of which are herein incorporated in their entirety.

### FIELD OF INVENTION

The present invention relates to devices, kits and methods for the rapid characterization of biomolecules recovered directly from biological samples. The devices, kits and methods according to the present invention summarily provide the basis for mass spectrometric immunoassays (MSIA), which are able to qualitatively and quantitatively analyze specific proteins, and their variants, present in a variety of biological fluids and extracts. Such MSIA devices, kits and methods have significant application in the fields of: basic research and development, proteomics, protein structural characterization, drug discovery, drug-target discovery, therapeutic monitoring, clinical monitoring and diagnostics, as well as in the high throughput screening of large populations to establish and recognize protein/variant patterns that are able to differentiate healthy from diseased states.

### BACKGROUND OF THE INVENTION

With the recent first draft completion of the human genome, much attention is now shifting to the field of proteomics, where gene products (proteins), their variants, interacting partners and the dynamics of their regulation and processing are the emphasis of study.

Such studies are essential in understanding, for example, the mechanisms behind genetic/environmentally induced disorders or the influences of drug mediated therapies, as well as potentially becoming the underlying foundation for further clinical and diagnostic analyses. Critical to these studies is the ability to qualitatively determine specific variants of whole proteins (i.e., splice variants, point mutations, posttranslationally modified versions, and environmentally/therapeutically-induced modifications) and the ability to view their quantitative modulation. Moreover, it is becoming increasingly important to perform these analyses from not just one, but from multiple biological fluids/extracts obtained from a single individual.

There are several challenges inherent to the analysis of these proteins, or for that matter, all proteins in general. The greatest challenge is the fact that any protein considered relevant enough to be analyzed resides in vivo in a complex biological environment or media. The complexity of these biological media present a challenge in that, oftentimes, a protein of interest is present in the media at relatively low levels and is essentially masked from analysis by a large abundance of other biomolecules, e.g., proteins, nucleic acids, carbohydrates, lipids and the like. In other instances, (e.g., the lipoproteins), proteins are complexed tightly with other biomolecules that might interfere with their analysis.

These analytical challenges are further exacerbated when the enormous breadth in genetic and posttranslational diversity residing in natural populations is taken into consideration. Essentially, any protein can take on numerous forms in populations dependent on slight differences in genetic code, posttranslational processing or even the biological medium in which the protein is present. Historically, these differences, once found, rigorously characterized and applied in clinical study have often been found to be the cause or diagnostic signal of disease. Multiple analytical approaches, including DNA and protein sequencing and immunological approaches such as ELISA and RIA, are generally needed to accurately determine the presence and identity of wide numbers of protein variants that reside in populations. However, when any one of these approaches is subsequently used in diagnostic applications, it is either tuned into a detection of specific variant or broadly detects all variants as a single species. In either case, the approach loses its ability as a discovery tool when applied diagnostically—essentially, by ignoring the presence of other variants.

Thus, in order to analyze proteins of interest from- and in- their native environment, assays capable of assessing proteins present in a variety of biological fluids and/or extracts, both qualitatively and quantitatively, are needed. Importantly, these assays must: 1) be able to selectively retrieve and concentrate specific proteins/biomarkers from various biological fluid/extract for subsequent high-performance analyses, 2) be able to quantify targeted proteins, 3) be able to recognize variants of targeted proteins (e.g., splice variants, point mutations, posttranslational modifications and environmentally /therapeutically induced chemical modifications) and to elucidate their nature, 4) be capable of analyzing for, and identifying, ligands interacting with targeted proteins, and 5) be able to analyze the same protein from multiple fluids/extracts taken from a single individual. Moreover, it is of great value to apply such analyses in high throughput manner to large numbers of samples in order to determine a statistical “normal” profile for any given protein in any particular fluid/extract from which “abnormal” differences are readily recognizable. Causes of such abnormalities may be related to genetic makeup, disease, therapeutic treatments or environmental stresses.

In order to accomplish such assays, it is necessary to combine selective purification/concentration approaches with analytical techniques capable of multi-protein detection and the rigorous structural characterization of biomolecules. One such approach is mass spectrometric immunoassay (MSIA), where affinity isolation is used in combination with matrix-assisted laser desorption/ionization time-of-flight mass spectrometry (MALDI-TOF MS) to form a concerted, high-performance technique for the analysis of proteins as disclosed in Nelson et al, Anal. Chem 1995 which is herein incorporated by reference. Utilizing this approach, a single pan-antibody can be used to retrieve all variants of a specific protein from a biological fluid, upon which each variant is detected during mass spectrometry at a unique and characteristic molecular mass. Moreover, resolution of related protein variants also allows mass-shifted variants of a target protein to be intentionally incorporated into the analysis for use as an internal reference standard (IRS) for quantitative analysis. Applied differently, the inherent resolution of MALDI-TOF MS allows the design of assays using multiple affinity ligands to selectively purify/concentrate and then analyze multiple proteins in a single assay. Overall, the MSIA approach can be used for the unambiguous detection and rigorous quantification of proteins and variants retrieved from complex biological systems. To date, however, approaches such as MSIA have not been driven in the breadth or capacity needed to make a significant impact in the biological sciences. Specifically, devices, kits and methods for the analysis of large numbers of selected proteins present in multiple biological fluids/extracts (in large numbers of individuals) are lacking.

For these foregoing reasons, there is a pressing need for rapid, sensitive and accurate analytical MSIA devices and analytical protocols for the analysis of proteins and their variants. This present application considers the proteins: orosomucoid 1, alpha-1-antitrypsin, alpha-1-antichymotrypsin, creatine kinase muscle/brain, cardiac troponin I, ceruloplasmin, plasminogen, ferritin light chain, lactoferrin, myoglobin, apolipoprotein CI, apolipoprotein CII, apolipoprotein CIII, and anti-thrombin III, present in various biological fluids/extracts found in individuals (humans). Moreover, there is a need to correlate the results of analyses performed using these assays with disease states in order to employ empirical findings in further applications such as drug and drug-target discovery, clinical monitoring and diagnostics.

Although the present invention has been described in some detail by way of illustration and example for purposes of clarity and understanding, it will be apparent that certain changes and modifications may be practiced within the scope of the appended claims.

### SUMMARY OF THE INVENTION

It is an object of the present invention to devise MSIA methods that are able to prepare micro-samples for mass spectrometry directly from biological fluid.

It is another object of the present invention to construct pipettor tips (termed MSIA-Tips) containing porous solid supports that are constructed, covalently derivatized with affinity ligand, and used to extract specific proteins and their variants in preparation for mass spectrometry.

Yet another object of the present invention is to apply in use the aforementioned MSIA methods and devices in analyzing specific proteins and their variants from biological fluids and extracts.

Another object of the present invention is to provide for protein and variant quantification using MSIA by ensuring the presence of a second protein species in the assay to serve as an IRS.

It is yet another object of the present invention to provide MSIA assays that have adequate quantitative dynamic ranges, accuracies, and linearities to cover the concentrations of proteins expected in the biological fluids.

A further object of this present invention is to provide useful product kits for the detection, qualification, and quantification of specific proteins and variants present in a variety of biological fluids or extracts obtained from a single individual.

It is still another object of the present invention to devise MSIA product kits for the analysis of the following proteins, and their variants, present in various biological fluids/extracts found in individuals (humans): orosomucoid 1, alpha-1-antitrypsin, alpha-1-antichymotrypsin, creatine kinase muscle/brain, cardiac troponin I, ceruloplasmin, plasminogen, ferritin light chain, lactoferrin, myoglobin, apolipoprotein CI, apolipoprotein CII, apolipoprotein CIII, and anti-thrombin III.

Yet a further object of the present invention is to use the aforementioned kits, devices and methods to detect variants of the target proteins.

Another object of the present invention is to use the methods, devices and kits of the present invention in the fields of basic research and development, proteomics, protein structural characterization, drug discovery, drug-target discovery, therapeutic monitoring, clinical monitoring and diagnostics.

It is still a further objective of the present invention to use the MSIA kits, devices and methods of the present invention in general population screens, which include both diseased and healthy-state individuals, to recognize and establish protein and variant patterns that correlate with disease.

The present invention includes the ability to selectively retrieve and concentrate specific biomolecules from biological fluid for subsequent high-performance analyses (e.g. MALDI-TOF MS), the ability to identify targeted biomolecules, the ability to quantify targeted biomolecules, the ability to recognize variants of targeted biomolecules (e.g., splice variants, point mutations and posttranslational modifications) and to elucidate their nature, and the capability to analyze for, and identify, ligands interacting with targeted biomolecules. The novel features that are considered characteristic of the invention are set forth with particularity in the appended claims. The invention itself, however, both as to its structure and its operation together with the additional objects and advantages thereof will best be understood from the following description of the preferred embodiment of the present invention when read in conjunction with the accompanying drawings. Unless specifically noted, it is intended that the words and phrases in the specification and claims be given the ordinary and accustomed meaning to those of ordinary skill in the applicable art or arts. If any other meaning is intended, the specification will specifically state that a special meaning is being applied to a word or phrase. Likewise, the use of the words “function ” or “means” in the Description of Preferred Embodiments is not intended to indicate a desire to invoke the special provision of 35 U.S.C. §112, paragraph 6 to define the invention. To the contrary, if the provisions of 35 U.S.C. §112, paragraph 6, are sought to be invoked to define the invention(s), the claims will specifically state the phrases “means for” or “step for” and a function, without also reciting in such phrases any structure, material, or act in support of the function. Even when the claims recite a “means for” or “step for” performing a function, if they also recite any structure, material or acts in support of that means of step, then the intention is not to invoke the provisions of 35 U.S.C. §112, paragraph 6. Moreover, even if the provisions of 35 U.S.C. §112, paragraph 6, are invoked to define the inventions, it is intended that the inventions not be limited only to the specific structure, material or acts that are described in the preferred embodiments, but in addition, include any and all structures, materials or acts that perform the claimed function, along with any and all known or later-developed equivalent structures, materials or acts for performing the claimed function.

### BRIEF DESCRIPTION OF THE DRAWINGS

FIG. 1 is a schematic illustration of the MSIA procedure.

FIG. 2 is an illustration of MSIA analysis of orosomucoid 1 (ORM1) from human plasma.

FIG. 3 is an illustration of MSIA analysis of orosomucoid 1 (ORM1) from human urine.

FIG. 4 is an illustration of MSIA analysis of Alpha-1-antitrypsin (AAT) from human plasma.

FIG. 5 is an illustration of MSIA analysis of Alpha-1-antitrypsin (AAT) from human unne.

FIG. 6 is an illustration of MSIA analysis of alpha-1-antichymotrypsin (ACT) from human plasma.

FIG. 7 is an illustration of MSIA analysis of creatine kinase muscle/brain (CK-MB) from human plasma.

FIG. 8 is an illustration of MSIA analysis of Cardiac Troponin I (cTnI) from human plasma.

FIG. 9 is an illustration of MSIA analysis of ceruloplasmin (CP) from human plasma.

FIG. 10 is an illustration of MSIA analysis of plasminogen (PSM) from human plasma.

FIG. 11 is an illustration of MSIA analysis of ferritin light chain (FTL) from human plasma and urine.

FIG. 12 is an illustration of MSIA analysis of lactoferrin (LTF) from human saliva.

FIG. 13 is an illustration of MSIA analysis of myoglobin (MYO) from human plasma samples obtained from two individuals, and using rabbit myoglobin as internal reference standard (IRS).

FIG. 14 is an illustration of multiplexed MSIA analysis of apolipoproten C's (ApoCI, ApoCII, ApoCIII) from human plasma.

FIG. 15 is an illustration of MSIA analysis of antithrombin-III (ATIII) from human plasma.

### DETAILED DESCRIPTION

The present invention provides for methods, devices and kits for the MSIA analysis of specific proteins and variants present in various biological fluids and extracts. Such analyses are essential for the detailed, full-length characterization of proteins as a function of the biological fluid/extract from which they originate. Proteins of concern in the present invention are orosomucoid 1, alpha-1-antitrypsin, alpha-1-antichymotrypsin, creatine kinase muscle/brain, cardiac troponin I, ceruloplasmin, plasminogen, ferritin light chain, lactoferrin, myoglobin, apolipoprotein CI, apolipoprotein CII, apolipoprotein CIII, and anti-thrombin III.

Another embodiment of the present invention provides for methods used in the quantification of proteins and variants present in various biological fluids. In certain analyses, multiple proteins/variants were simultaneously retrieved from a given biological fluid/extract. The target proteins were then quantified (either relative or absolute) by equating relative signal intensities/integrals with analyte concentration.

Yet another embodiment of the present invention provides for the use of MSIA in screening of individuals in large populations for specific proteins and variants present in various biological fluids. When applied to multiple individuals, the MSIA assays are able to yield intra-individual and inter-individual details, regarding a specified protein, which upon correlation can be linked to genetic, transcriptional or posttranslational differences, disease state, response to therapy or environmental stress, or differences in metabolism/catabolism.

In another embodiment, the present invention is used to discover new protein variants that are linked to either healthy or disease states. During analyses, different variants of the target proteins are observed dependent on the biological fluid or individual from which they were retrieved. The differences observed using the MSIA approach form the basis for clinical or diagnostic assays.

Specific embodiments in accordance with the present invention will now be described in detail using the following lexicon. These examples are intended to be illustrative, and the invention is not limited to the materials, methods or apparatus set forth in these embodiments.

As used herein, “MSIA-Tips” refers to a pipettor tip containing an affinity reagent.

As used herein, “affinity ligand” refers to atomic or molecular species having an affinity towards analytes present in biological mixtures. Affinity ligands may be organic, inorganic or biological by nature, and can exhibit broad (targeting numerous analytes) to narrow (target a single analyte) specificity. Examples of affinity ligands include, but are not limited to, receptors, antibodies, antibody fragments, synthetic paratopes, enzymes, proteins, multi-subunit protein receptors, mimics, chelators, nucleic acids, and aptamers.

As used herein, “analyte” refers to molecules of interest present in a biological sample. Analytes may be, but are not limited to, nucleic acids, DNA, RNA, peptides, polypeptides, proteins, antibodies, protein complexes, carbohydrates or small inorganic or organic molecules having biological function. Analytes may naturally contain sequences, motifs or groups recognized by the affinity ligand or may have these recognition moieties introduced into them via chemical or enzymatic processes.

As used herein, “biological fluid or extract” refers to a fluid or extract having a biological origin. Biological fluid may be, but are not limited to, cell extracts, nuclear extracts, cell lysates or biological products used to induce immunity or substances of biological origin such as excretions, blood, sera, plasma, urine, sputum, tears, feces, saliva, membrane extracts, and the like.

As used herein, “internal reference standard” refers to analyte species that are modified (either naturally or intentionally) to result in a molecular weight shift from targeted analytes and their variants. The IRS can be endogenous in the biological fluid or introduced intentionally. The purpose of the IRS is that of normalizing all extraction, rinsing, elution and mass spectrometric steps for the purpose of quantifying targeted analytes and/or variants.

As used herein, “posttranslational modification” refers to any alteration that occurs after synthesis of the polypeptide chain. Posttranslational modifications may be, but are not limited to, glycosylation, phosphorylation, sulphation, amidation, cysteinylation, dimerization, or enzymatic or chemical additions or cleavages. The cause of the posttranslational modifications can be endogenous (e.g., systematic within the individual), environmentally or therapeutically induced or in response to external stimuli such as stress or infection (e.g., bacterial or viral).

As used herein, “genetic difference” refers to differences in nucleic acid sequence (e.g., DNA or RNA) that result in a recognizable mass shift on the protein level. Genetic differences may be nucleotide polymorphisms, variations in short tandem repeats, variations in allele, or transcriptional variations (e.g., splice variants).

As used herein, “wild-type” refers to the variation of a given protein most commonly found in nature. The wild-type protein is generally recognized as the functional form of the protein, including all transcriptional and posttranslational processing. The wild-type protein can be found empirically using MSIA by assaying large numbers of individuals and determining the high-percentage variant.

As used herein, “variant” refers to different forms of a given protein resulting from genetic differences or posttranslational modifications. As generally applied, MSIA recognizes the variants by observing them as signals mass-shifted from those expected for the wild-type protein.

As used herein, “mass spectrometer” refers to a device able to volatilize/ionize analytes to form vapor-phase ions and determine their absolute or relative molecular masses.

Suitable forms of volatilization/ionization are laser/light, thermal, electrical, atomized/sprayed and the like or combinations thereof. Suitable forms of mass spectrometry include, but are not limited to, Matrix Assisted Laser Desorption/Time of Flight Mass Spectrometry (MALDI-TOF MS), electrospray (or nanospray) ionization (ESI) mass spectrometry, or the like or combinations thereof.

### EXAMPLE 1

### General Msia Method

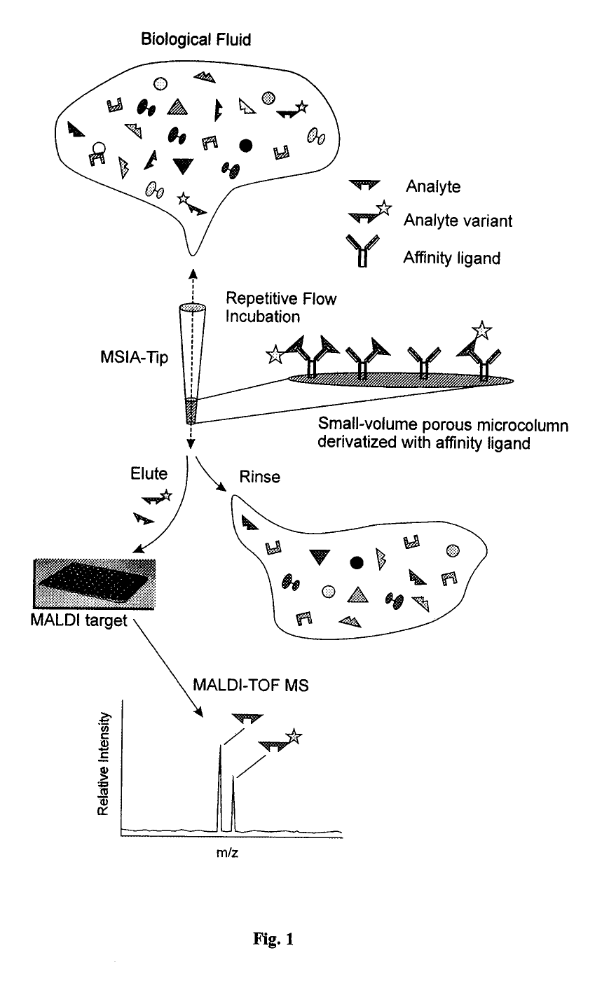

The general MSIA approach is shown graphically in FIG. 1. MSIA-Tips contain porous solid supports covalently derivatized with affinity ligands that are used to extract the specific analytes and their variants from biological samples by repetitively flowing the samples through the MSIA-Tips. Once washed of non-specifically bound compounds, the retained analytes are eluted onto a mass spectrometer target using a MALDI matrix.

MALDI-TOF MS then follows, with analytes detected at precise m/z values. The analyses are qualitative by nature but can be made quantitative by incorporating mass-shifted variants of the analyte into the procedure for use as internal standards.

With regard to the proteins listed in the following EXAMPLES, mass spectrometric immunoassays were performed in the following general manner (additional methodologies specific to each protein are addressed when needed in the Examples):

The MSIA-Tips used in urine and blood analyses were constructed having a single-piece (monolithic—acting both as a stationary phase and derivatizable support) porous micro-frit (0.25-2.5 μL dead volume) at the entrance to a microcolumn with adequate volume (10-1000 μL) to accommodate the volume of the sample. The microcolumn barrels were constructed from glass or plastic and in the form of tapered or straight capillaries or pipettor tips. The porous microfrits were manufactured using any number of derivatization schemes that ultimately result in free functional groups able to be activated for subsequent coupling of antibodies/affinity ligands via covalent linkage through amines, carboxylic acids or sulfhydryls. Antibodies were monoclonal or polyclonal and were prepared from the serum of inoculated organism (e.g., rabbit, mouse, goat or other antibody-producing organism), or ascites fluid via Protein A/G extraction or affinity purification prior to linkage to the MSIA-Tips. Other affinity ligands were isolated/prepared using similar affinity and standard chromatographic approaches.

For analysis from blood, a 50 μL sample of human whole blood was collected from a lancet-punctured finger using a capillary microcolumn (heparinized, EDTA, or no coating) and mixed with 200 μL of HBS buffer (10 mM HEPES, 150 mM NaCl, pH 7.4, 3 mM EDTA, 0.005% polysorbate 20 (v/v)) and centrifuged for 30 seconds (at 7,000 RPM, 3000×g) to pellet the red blood cells. Aliquots (10-220 μL) of the supernatant (diluted plasma) were subsequently mixed with additional HBS buffer to bring the total volume of the diluted plasma to 400-1200 μL. Analyses were performed from a diluted plasma sample by repeatedly (5-500 cycles, 20-200 μL/cycle, 10-100 cycles/minute) drawing and expelling the sample through an antibody-derivatized affinity microcolumn (MSIA-Tips).

After selective extraction/concentration of the specified protein, tips were rinsed (with e.g., water, buffers, detergents, organic solvents or combinations thereof) to remove traces of non-specifically retained compounds. Retained compound were eluted for MALDI-TOF MS using a small volume (0.5-5 μL) of a chaotrope and then adding a common MALDI matrix solution (e.g., α-cyano-4-hydroxycinnamic acid or sinapinic acid in acetonitrile/water/trifluoroacetic acid mixture), or simply by using a MALDI matrix solution. MALDI-TOF MS was performed using linear delayed-extraction mass spectrometer, although other forms of MALDI mass spectrometers could be used.

Oftentimes, multiple MSIA analyses were performed serially from a single plasma sample by addressing the sample with a first antibody-derivatized MSIA-Tip followed by subsequent tips (e.g., a second tip specific to a second protein, a third tip specific to a third protein, etc). This approach increased the efficiency of use of a single sample and resulted in the need to draw less blood from an individual.

Analyses were performed from urine using an approach similar to that described for plasma. Urine samples were prepared for analysis by addition of a pH compensating buffer such as 2M ammonium acetate (pH=7.6) that contained a protease inhibitor cocktail. Additionally, because of its availability, and generally lower concentration of target proteins, larger volumes (0.2-50 mL) of urine were addressed. To ensure complete incubation of the larger volumes with the MSIA-Tips, a larger number of incubation cycles (100-1000) were used. Rinse, elution, preparation and MALDI-TOF MS protocols were the same as for plasma analyses.

Oftentimes, multiple proteins were analyzed from a single urine sample in parallel by addressing the sample with parallel repeating robotics fitted with multiple MSIA-Tips, each targeting a different protein. This approach required less time spent for each analysis, as well as made more efficient use of a sample.

### EXAMPLE 2

### Orosomucoid 1 (ORM1)

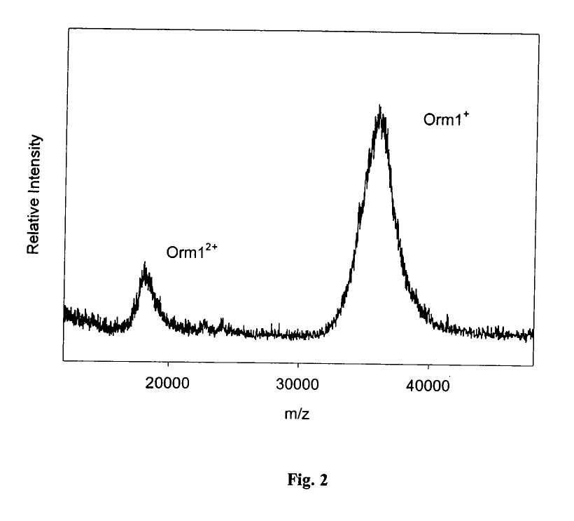

Orosomucoid 1 (ORM1) is a heavily glycosylated (˜45% carbohydrate content) serum protein that has been indicated as a putative biomarker for a number of inflammatory (acute-phase) ailments. ORM1 exhibits large heterogeneity in structure due to the heterogeneity of the carbohydrate chain and genetic polymorphisms. FIG. 2 shows the results of ORM1-MSIA performed on the plasma of a healthy individual. Briefly, a 50 μL sample of whole blood was collected from a lancet-punctured finger using a heparinized microcolumn, mixed with 200 μL HBS buffer and centrifuged for 30 seconds (at 7,000 RPM, 3000×g) to pellet the red blood cells. A 200 μL volume of the supernatant was then subjected to MSIA by repeatedly (50 times, 100 μL each time) aspiring and dispensing the medium through an anti-ORM1 MSIA-Tip (made by linking polyclonal anti-ORM1 antibody onto carboxymethyldextran (CMD)-modified solid support (within the MSIA-Tip) via 1,1′-carbonyldiimidazole (CDI)-mediated coupling). After extraction, the tip was rinsed with HBS (10 aspirations and dispensing) followed by water (10×100 μL). The captured proteins were eluted from the MSIA-Tip with a small volume of MALDI matrix (saturated aqueous solution of sinapinic acid (SA), in 33% (v/v) acetonitrile, 10% (v/v) acetone, 0.4% (v/v) trifluoroacetic acid) and stamped onto a MALDI target array surface comprised of self-assembled monolayers chemically masked to make hydrophilic/hydrophobic contrast target arrays. The sample spot on the target array was analyzed using MALDI-TOF mass spectrometry. The resulting mass spectrum (FIG. 2) shows a strong signal centered at m/z ˜36,000 corresponding to the singly-charged ORM1. Unlike other, non-glycosylated proteins in the same molecular weight region, the ion signal of the ORM1 is exceptionally broad, indicating that the ORM1 exists in plasma as a group of highly dispersed glycoforms. A doubly charged ORM1 signal is also observed.

ORM1 was also analyzed directly from the urine of the same individual using a similar procedure. Briefly, 30 mL of urine (fresh, mid-stream void) was collected from the individual and mixed with 30 mL HBS buffer (1:1 ratio). ORM1 was selectively extracted from the diluted urine by repeatedly (300 times, 200 μL each time) aspiring and dispensing the sample through an anti-ORM1 MSIA-Tip (made in the same way as described above).

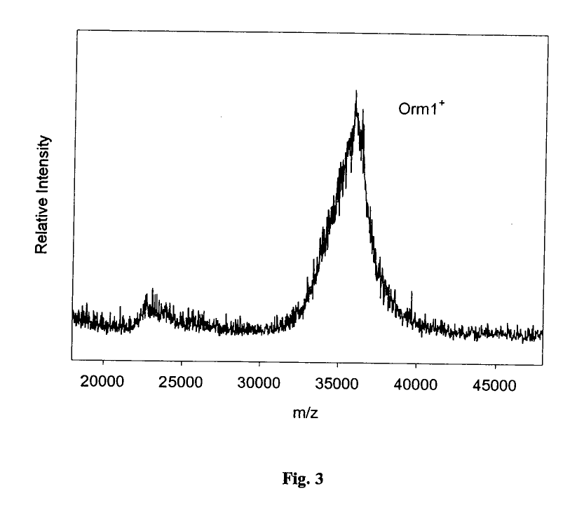

After extraction, the tip was treated as described above, and the matrix/protein eluant analyzed by MALDI-TOF MS. FIG. 3 shows the results of the ORM1-MSIA analysis. Similar to the plasma spectrum, the urine spectrum shows a strong broad signal centering at m/z ˜35,400, corresponding to the singly-charged ORM1.

### EXAMPLE 3

### ALPHA-1-ANTITRYPSIN (AAT)

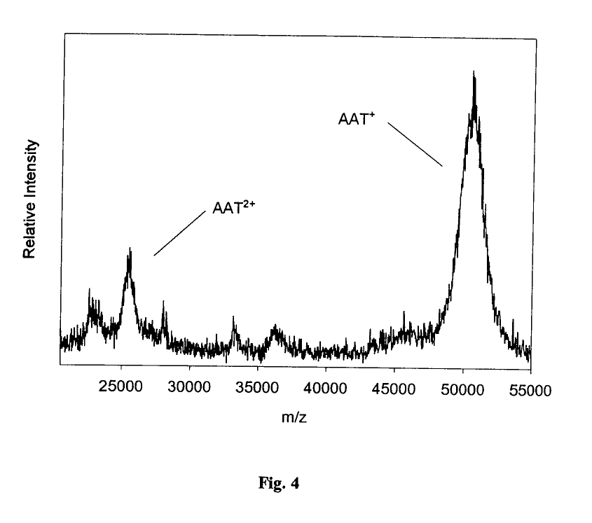

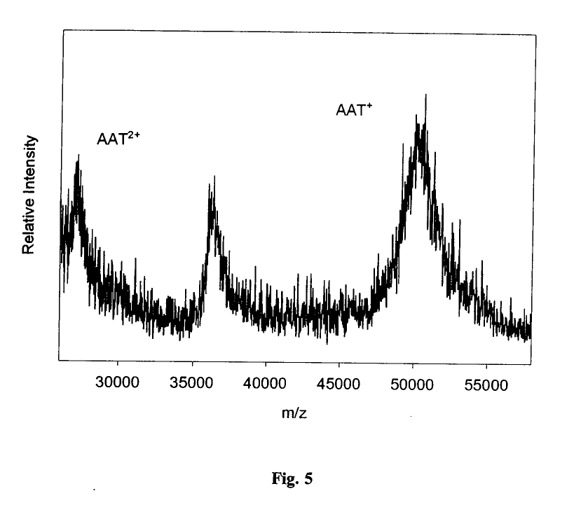

Alpha-1-antitrypsin (AAT) is a moderately glycosylated glycoprotein having inhibitory action towards the serine protease trypsin. Additionally, AAT is highly polymorphic, existing in human populations in at least four major allelic variants. Aside from the high-frequency allelic variants, certain polymorphisms (point mutations) have been linked with emphysema and certain liver disorders. MSIA analysis of AAT from plasma, performed as described in EXAMPLE 2, shows a strong, broadened ion signal at m/z ˜51,000 reflecting polymorphic and glycosylation variants (FIG. 4). The analysis of AAT directly from the urine of the same individual (see protocol in Example 2) yields similar results with regard to parent ion signal of the AAT (FIG. 5). Interestingly, an additional signal at m/z ˜23,900 is observed in the mass spectrum from the AAT-MSIA plasma analysis (FIG. 4). Although the identity of the 23.9 kDa signal has yet to be determined, a possible candidate is tryspin (molecular mass of 23.9 kDa) that enters into the analysis via in vivo binding to the AAT. Given that this possibility holds true, the AAT-MSIA stands to find use in determining biological activity of AAT (in individuals) by observing both species simultaneously in the same analysis.

### EXAMPLE 4

### Alpha-1-Antichymotrypsin (ACT)

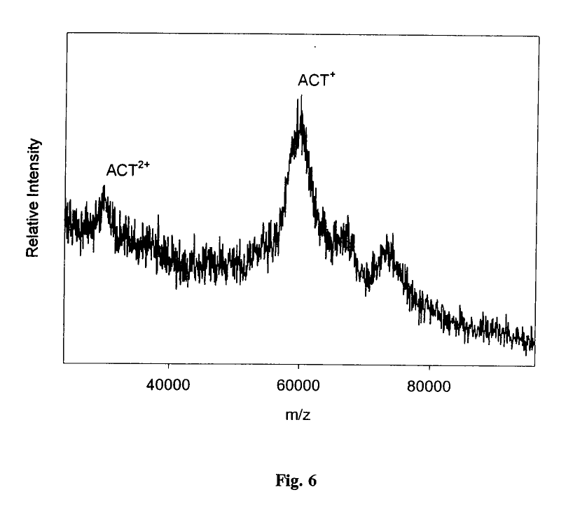

Alpha-1-antichymotrypsin (ACT) is a serine proteinase inhibitor that forms enzymatically inactive complex with its target proteinases, in specific alpha-chymotrypsin and cathepsin G. ACT is synthesized in the liver, contains 398 amino acid residues, two N-linked carbohydrate chains, and, like AAT, its concentration increases in the acute phase of inflammation or infection. The result of the MSIA analysis of ACT from plasma, performed as described in EXAMPLE 2, is shown in FIG. 6. A major peak at ˜59 kDa, characteristic of the glycosilated ACT is observed in the mass spectrum.

### EXAMPLE 5

### Creatine Kinase Muscle/Brain (CK-MB)

Creatine kinases are tissue/organ-specific dimeric isoenzymes, which although having the same biological function, have slightly different amino acid sequences dependent on the tissue/organ from which they originate. Historically, CK-MM (muscle-muscle dimer) has served as a quantitative biomarker for severe myocardial infarction (MI). Essentially, CK-MM levels in the blood stream are significantly elevated upon trauma suffered to the heart during MI. However, normal plasma also contains a significant level of CK-BB (brain-brain dimer) and thus assays for CK-MM or CK-MB (the muscle-brain isoenzyme dimer) must be highly specific in order to differentiate between the different tissue/organ-specific forms of the dimeric isoenzyme. To date, there is no high-specificity assay that is able to resolve the tissue/organ-specific forms of CK in a single analysis.

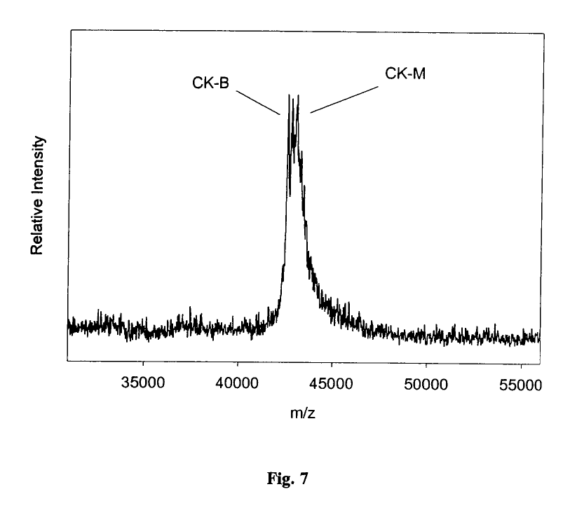

FIG. 7 shows the results of a CK-MSIA, performed as described in EXAMPLE 2, applied to plasma taken from an individual suffering from cardiac complications. Two dominant signals are present in the mass spectrum at m/z=42,506 and m/z=42,978. The observed difference stems from slight differences in amino acid sequence between the two forms, which taken collectively result in a 456.9 Da shift in molecular mass between the muscle (MW_{calc}=42,965.9) and brain (MW_{calc}=42,509.0) isoforms of the enzyme. As is readily apparent, the MSIA approach is able to detect each isoform as a separate signal in the single mass spectrum. The result is a single assay readily able to differentiate the diagnostic form of CK (CK-M) from the non-diagnostic form (CK-B). Such an assay stands to have use in the study and diagnosis of cardiovascular disease.

### EXAMPLE 6

### Cardiac Troponin I (cTnI)

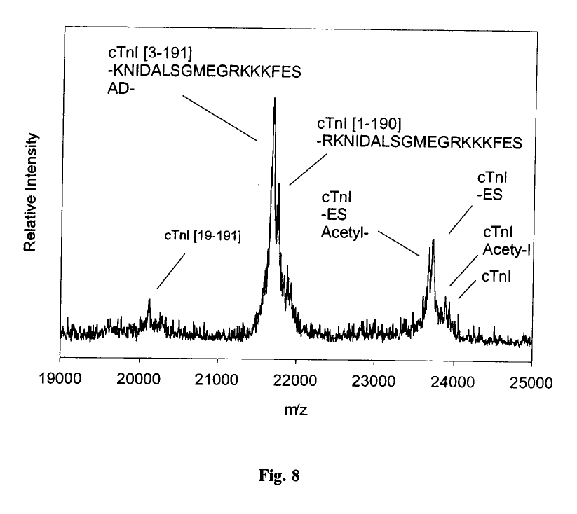

Cardiac Troponin I (cTnI) is another quantitative biomarker generally used in monitoring cardiovascular health. The circulating level of cTnI is a specific marker for myocardial infarction (MI), since cTnI is rapidly released (within 2-3 hours) into serum/plasma at onset. The levels of cTnI are generally monitored using immunometric approaches (ELISA or RIA). FIG. 8 shows the results a cTnI-MSIA, performed as described in EXAMPLE 2, applied to the same plasma used in EXAMPLE 5. Observed in the spectrum are signals with m/z=23,923, 23,884, 23,703, 23,665, 21,739, 21,666 and 20,104. These signals correspond with N-terminally acetylated full-length cTnI (residues 1-209 MW_{calc}=23,916.3) and a first truncated variant of cTnI (residues 1-207; MW_{calc}=23,700.1). These peaks are accompanied by signals that are m/z=˜42 smaller, which correspond to the full-length cTnI and the first truncated variant lacking the N-terminal acetylation. The next signals correspond to significantly truncated cTnI (residues 1-190, MW_{calc}=21, 739.8; residues 3-191; MW_{calc}=21,667.8, and residues 19-191; MW_{calc}=20094.1). The proteolytic removal of the C-terminal 19 amino acid of cTnI has been found to occur during MI. The presence of N- and C-terminal truncations has been previously identified, but have not been mass spectrometrically characterized. The MSIA approach is able to augment these existing approaches by determining that the cardiac marker is in fact seven different versions of the same protein rather than the assumed single intact protein.

### EXAMPLE 7

### Ceruloplsamin (CP)

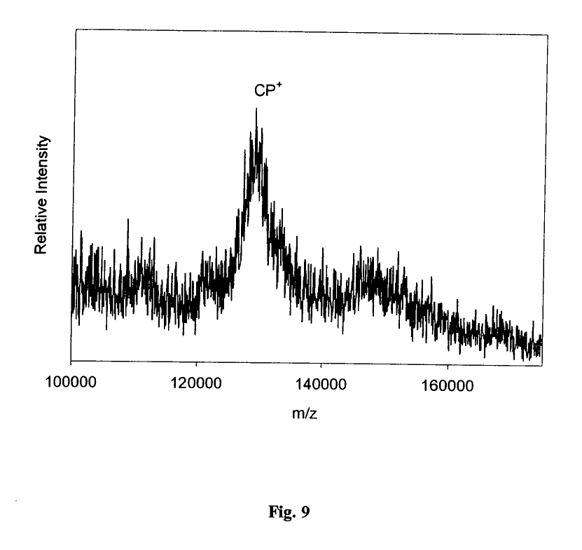

Ceruloplasmin (CP) is a copper oxidase enzyme that serves in the maintenance of heptatic copper homeostasis. Active CP levels in plasma are decreased in Wilson's disease and Menke's disease, both characterized by the poor uptake of dietary copper. Ceruloplasmin levels are increased in infection, inflammatory diseases, and neoplastic diseases. FIG. 9 shows the results of a CP-MSIA performed on a human plasma sample as described in EXAMPLE 2. Observed in the spectrum is a broad signal at m/z ˜128,000, representing singly charged glycosilated CP.

### EXAMPLE 8

### Plasminogen (PSM)

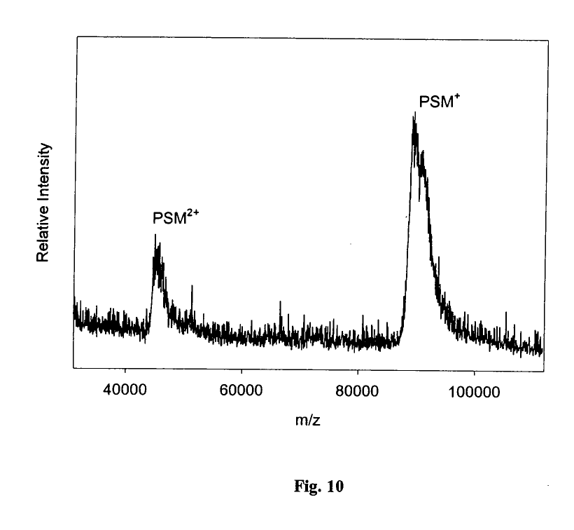

Plasminogen (PSM) is the inactive precursor of the blood clot-dissolving enzyme, plasmin. Plasminogen is found incorporated into blood clots at high concentrations, and circulating at (relatively) much lower concentrations. FIG. 10 shows the results of a PSM-MSIA performed on human plasma as described in EXAMPLE 2. The spectrum is dominated by the singly-charged plasminogen signal at m/z ˜90,000. Closer inspection of the signal reveals the presence of at least two high dispersity forms of the plasminogen. The different forms are likely due to macro- and microheterogeneity in the glycosylation pattern of at least two glycosylation sites present on the backbone protein.

### EXAMPLE 9

### Ferritin Light Chain (FTL)

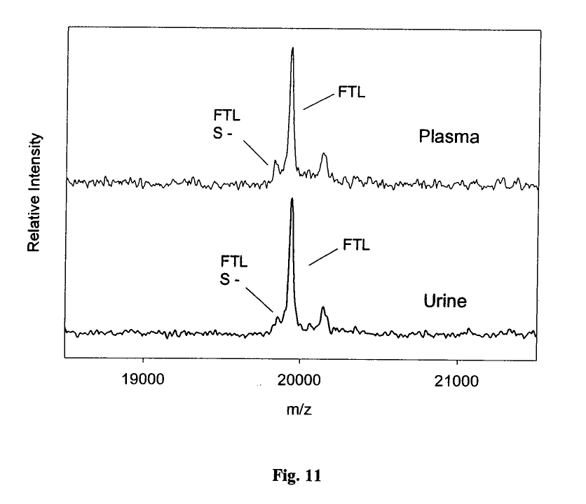

Ferritin is the major intracellular iron storage protein in all organisms. It is comprised of 24 subunits of ferritin heavy (FTH1) and light chains (FTL) and is present in virtually all cells, and at low concentrations in plasma. FIG. 11 shows the results of a FTL-MSIA performed from human plasma (upper trace) and urine (lower trace), as described in EXAMPLE 2. Dominating the mass spectrum is a signal from the intact FTL, which is N-terminally acetylated, and a minor signal from the loss of N-terminal Serine. Interestingly, the truncated variant is observed at a mass reflective of the loss of only Ser, not Acetyl-Ser, suggesting that the truncated variant is formed by N-terminal cleavage of the intact precursor prior to global acetylation of all variants. In addition to finding use in studying the mechanism of FTL processing, this assay stands to find significant use screening individuals for hyperferritinemia and cataract formation associated with point mutations present in FTL.

### EXAMPLE 10

### Lactoferrin (LTF)

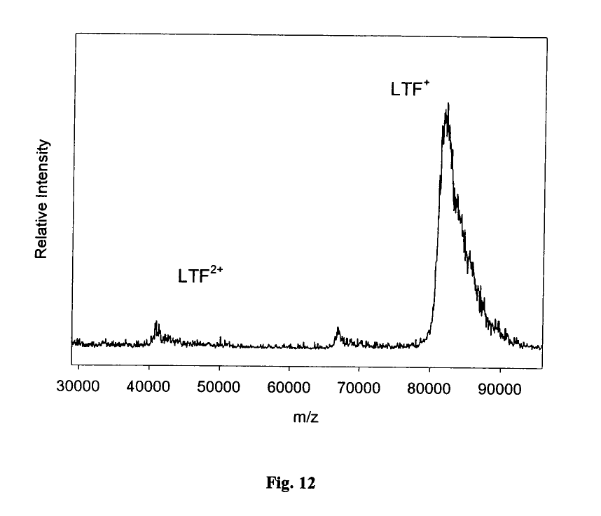

Lactoferrin (LTF) is a member of the transferrin family whose primarily function is that of iron transport in biological fluids. Lactoferrin has also been found to have moderate antiviral activity, and thus may serve the secondary role as an in vivo anti-microbial agent. FIG. 12 shows the results of a LTF-MSIA applied to human saliva—by substituting whole saliva for blood and following the procedure given in EXAMPLE 2. The presence of LTF, as a moderately glycosilated protein species, is indicated by the intense ion signal centering at ˜82 kDa.

### EXAMPLE 11

### Myoglobin (MYO)

The major function of myoglobin (MYO) in mammals is that of storing and transporting oxygen throughout muscle tissue. Basal levels of MYO in the blood stream are generally low, on the order of 0.05-0.1 mg/L. However, upon cardiac trauma myoglobin is immediately released in relatively large amounts into the blood stream, making it a potential “rapid-response ” marker for myocardial infarction (MI). Accordingly, a quantitative assay was constructed for human-MYO using rabbit-MYO as an internal reference standard, IRS (rabbit-MYO has a mass higher by 36.9 Da from human-MYO). Furthermore, the assay was designed and constructed as a “sight assay”, taking into account the normal variations in myoglobin concentrations in healthy individuals: the height of the MYO signal representing the maximum level of MYO found in healthy individuals was always lower than the height of the IRS signal. In this manner, the MYO-MSIA results can serve as an indicator able to immediately differentiate between cardiac trauma (which result in significantly elevated myoglobin levels) and fluctuating MYO levels (due to e.g., strenuous exercise) found in healthy individuals.

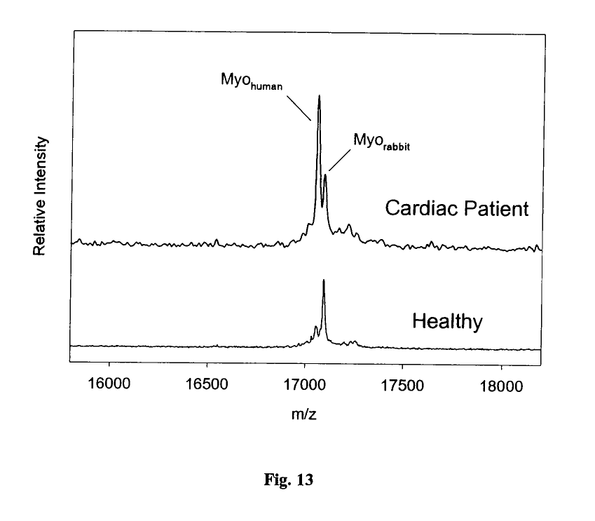

FIG. 13 shows the results of the quantitative MYO-MSIA applied to plasma samples from a healthy individual (lower trace) and an individual with elevated MYO levels due to heart trauma (upper trace). Each assay required ˜15 minutes to perform, and the assays were executed as described in EXAMPLE 2. Whereas the MYO signal from the healthy individual is observed to register in the normal range (below the IRS signal height), the MYO signal from the affected individual is observed at a level far above the normal range (estimated at >100-fold over normal, based on the fact that the plasma was diluted 100-fold prior to the MSIA so that the MYO signal is brought down in the same dynamic range as the IRS species). Such an assay stands to find use in biochemically differentiating symptomatic interferences (e.g., angina or hiatal hernia) from true cardiac trauma.

### EXAMPLE 13

### Apolipoprotein Cs' (ApoCI, ApoCII and ApoCIII)

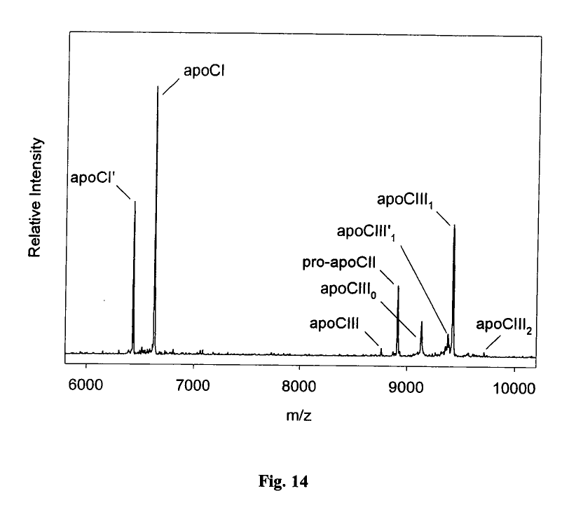

The apolipoprotein Cs' are small (˜6-9 kDa) polypeptides whose function is that of aiding in the transportation and metabolism of lipoproteins throughout the blood stream. FIG. 14 shows the results of a multiplexed MSIA designed to analyze the three apolipoprotein Cs', ApoCI, ApoCII, and ApoCIII (and their variants) in a single analysis. Briefly, MSIA-Tips were derivatized with a mixture of polyclonal antibodies that targeted all three of the major ApoC classes in a single assay. The devices were then used in analysis of plasma as described in EXAMPLE 2. Of particular note is the presence of multiple in vivo variants stemming from each of the ApoC “parent” species. These variants result from multiple post-translational modifications ranging from the loss of terminal end residues to the attachment of sugars (glycosylation). One variant observed in addition to the parent ApoCI (MW=6630.6) was the isoform (ApoCI′, MW=6432.4Da) created by the loss of the N-terminus Thr-Pro- from the parent. For ApoCII, only the pro-peptide form of the parent (pro-ApoCII, MW=8914.9, mature chain of ApoCII with a N-terminal hexapeptide) was observed. Multiple variants of ApoCIII differentiating with respect to glycosylations were observed. The first of the glycosylations is the attachment of one molecule of galactose and one molecule of N-acetyl-galactosamine to the parent (ApoCIII, MW=8764.7) producing apoCIII₀ (MW=9130.0). The attachments of 1 and 2 sialic acids to the glycan produce apoCIII₁ (MW=9421.3) and ApoCIII₂ (MW=9712.6), respectively. Furthermore, the ApoCIII₁ variant is truncated (the removal of the C-terminus Ala) to produce ApoCIII₁′(MW=9350.2). The nature of the variants suggests extensive posttranslational modification occurring on each of the parent species—ultimately reaching a blood-borne equilibrium that is observed generally throughout human populations. The MSIA-ApoCs assay is readily able to define such a “normal” equilibrium distribution (observed in healthy individuals) and detect/characterize shifts in the ApoCs distribution patterns associated with (e.g., cardiovascular) disease.

### EXAMPLE 14

### Anti-Thrombin III (ATIII)

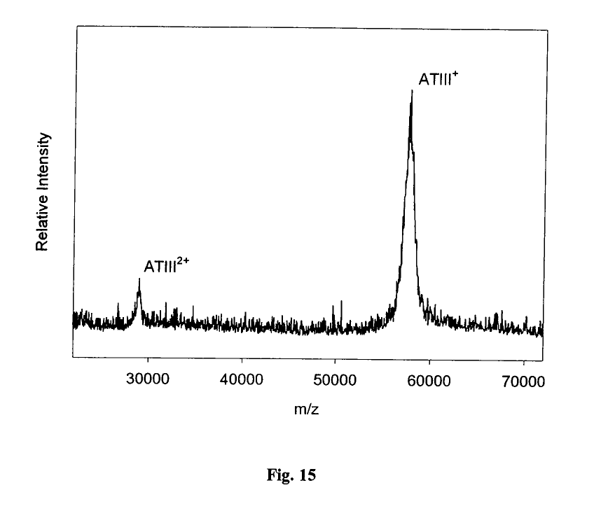

As the name implies, antithrombin III (ATIII) exhibits anticoagulation action by inhibiting the blood coagulation factor thrombin, as well as a number of other clotting factors. FIG. 15 shows the results of an ATIII-MSIA taken from human plasma, performed as described in EXAMPLE 2. Readily observed in the mass spectrum are singly and doubly charged signals from ATIII.

The present invention and the results shown in FIGS. 2 through 15 clearly demonstrate the usefulness of MSIA in the analysis of specific proteins and variants present in various biological fluids as well as the need for MSIA kits to expedite and enable the use of MSIA in analysis for specific proteins and variants present in various biological fluids. Generally, MSIA kits consist of devices, methods and reagents that facilitate rapid and efficient extraction of specific proteins and variants present in various biological fluids. Specifically, MSIA kits may consist of any or all of following items: MSIA-Tips, sample facilitating devices, samples, sample retaining/containment devices, activating reagents, affinity ligands, internal reference standards, buffers, rinse reagents, elution reagents, stabilizing reagents, mass spectrometry reagents and calibrants, mass spectrometry targets, mass spectrometers, analysis software, protein databases, instructional methods, specialized packaging and the like.

The preferred embodiment of the invention is described above in the Drawings and Description of Preferred Embodiments. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s). The foregoing description of a preferred embodiment and best mode of the invention known to the applicant at the time of filing the application has been presented and is intended for the purposes of illustration and description. It is not intended to be exhaustive or to limit the invention to the precise form disclosed, and many modifications and variations are possible in the light of the above teachings. The embodiments were chosen and described in order to best explain the principles of the invention and its practical application and to enable others skilled in the art to best utilize the invention in various other embodiments and with various modifications as are suited to the particular use contemplated.

We claim:

1. A method for quantifying the relative amount of one or more specific protein species present in a specimen, comprising the steps of:
a. combining said specimen with a known amount of internal reference species (IRS) if the specimen does not already contain one;
b. capturing and isolating plasminogen and said IRS, wherein said capturing and isolating step comprises a substep of combining said IRS containing specimen with an affinity reagent;
c. quantifying the plasminogen in which said quantifying step comprises using only mass spectrometric analysis to resolve distinct signals for the plasminogen and said IRS to determine the amount of the captured plasminogen relative to the IRS.

2. The method according to claim 1 in which said capturing and isolating step further comprises the steps of:
a. immobilizing at least one antibody onto a solid substrate to produce the affinity reagent;
b. combining an effective amount of the affinity reagent with the specimen to produce both a post-combination affinity reagent which includes the affinity reagent and a portion of the specimen containing the IRS and the plasminogen, and an unbound remainder of the specimen;
c. separating the post-combination affinity reagent from the unbound remainder of the specimen to form an isolated post-combination affinity reagent;
d. adding a laser desorption/ionization agent to the isolated post-combination affinity reagent to form a post-combination affinity reagent mass spectrometric mixture.

3. The method according to claim 2 in which said quantifying step further comprises the steps of:
a. mass spectrometrically analyzing the post-combination affinity reagent mass spectrometric mixture to produce a post-combination affinity reagent mass spectrum having a mass spectrometric response for the internal reference species located at a unique mass-to-charge ratio of the IRS, and a specific protein variant mass spectrometric response at a unique mass-to-charge ratio of the plasminogen thereby detecting the plasminogen and no mass spectrometric response corresponding to the mass-to-charge ratio of the plasminogen when the specimen contains no detectable amount of the plasminogen; and
b. determining whether the amount of the plasminogen present in the sample is greater or less than the known amount of the IRS by comparing the mass spectrometric response for detected plasminogen relative to the mass spectrometric response for the IRS.

4. The method of claim 2 further including the step of adding a chaotrope to the isolated post-combination affinity reagent prior to the adding laser desorption/ionization agent step.

5. The method of claim 3 further including the step of adding a chaotrope to the isolated post-combination affinity reagent prior to the adding laser desorption/ionization agent step.
